(19) United States
(12) Patent Application Publication
(10) Pub. No.: US 2015/0238226 A1
(43) Pub. Date: Aug. 27, 2015

(54) ZHUKOVSKY DOUBLE-BALLOON OBSTETRIC CATHETER

(71) Applicant: Yakov Grigoryevich ZHUKOVSKY, (US)

(72) Inventor: Yakov Grigoryevich Zhukovsky, Moscow (RU)

(21) Appl. No.: 14/427,059

(22) PCT Filed: Jul. 17, 2013
(86) PCT No.: PCT/RU13/00609

§ 371 Date: Mar. 10, 2015

(30) Foreign Application Priority Data
Sep. 10, 2012 (RU) 2012138566

(51) Int. Cl. A61B 17/42; A61M 25/10; A61B 17/12

(52) U.S. Cl. CPC A61B 17/42; A61B 17/12045; A61B 17/12099; A61B 17/12136; A61M 25/1011; A61B 2017/12004; A61B 2017/4216

(57) ABSTRACT

This invention relates to a double-balloon obstetric catheter. The double-balloon obstetric catheter includes a uterine balloon and a vaginal balloon that are provided, respectively, with a uterine-balloon axial tube and a vaginal balloon axial tube. The uterine-balloon axial tube is arranged coaxially within the vaginal-balloon axial tube with the possibility of fixing a change in a distance between the balloons along the longitudinal axis of the device. The vaginal balloon is attached to the distal end of the vaginal-balloon axial tube, and the shape of the distal end of the vaginal-balloon axial tube is adapted to the anatomical contours of the uterine neck. The catheter enables treatment of both prenatal and postnatal bleedings efficiently, as well as prevention of pathologic hemorrhages and infectious complications during caesarean sections.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### BACKGROUND OF THE INVENTION

1. Field of the Invention

The present invention relates to the field of medicine and medical devices, in particular, to the field of obstetrics, and, in particular, to a double-balloon obstetric catheter.

2. Description of Related Art Including Information Disclosed Under 37 CFR 1.97 and 37 CFR 1.98.

Postnatal bleedings are associated with primary causes of maternal mortality and morbidity, and, while this cause prevails in developing countries, bleedings during postnatal morbidity still are the leading cause thereof both in developing and developed countries. They are associated with severe conditions, such as acute renal insufficiency, necrosis of the adenohypophysis, hypoxic encephalopathy and coagulopathy.

During recent years, when bleeding cannot be stopped by administering uterotonic preparations, the world community of obstetricians recommend using balloon tamponade for the uterus (Georgiou C. “Balloon tamponade in the management of postnatal hemorrhage: a review”. BJOG. 2009 May; 116(6):748-57).

The efficiency of obstetric balloon catheters may be improved, provided several mechanisms for bleeding stopping are used, such as:

a mechanism of direct pressure on a damaged vessel, i.e., in a case of postnatal bleeding, on exposed placental site spiral arteries that are the direct cause of bleeding in a case of uterus atony, and

a mechanism of tourniquet, that is for stopping of a blood flow in vessels above the bleeding site, e.g., in uterus arteries due to their compression in the region of parametrium, which is, substantially, equal to bandaging or embolization of uterus vessels.

A method for treating postnatal uterine bleedings is known in the art, which is described in the publication by V. Ye. Radzinsky, Ya. G. Zhukovsky et al. “Treatment of Hypotonic Uterine Bleedings by Balloon Tamponade. A New Technology for an Old Method”. Akushetrstvo I Ginecologia, 2007, V. 1, p.48-49. The known method uses only the mechanism of direct pressure on a damaged vessel, where the wall of a filled balloon exerts pressure on open, bleeding spiral arteries of the placental site, that results in closing their walls, stopping bleeding, with subsequently forming thrombi in these vessels.

Uterine balloons structures and methods of their use are described in the literature (see, e.g., U.S. Pat. No. 6,676,680 by Packer, Jan. 13, 2004; CN No. 2768680 by Song, Apr. 4, 2006). Thus, e.g., CN Patent No. 201192357 by Fang, Nov. 2, 2009, described a balloon for filling with air that has valves and a pressure gage. Also, balloons for filling with a liquid are known (RU 66665 U1 by Zhukovsky et al, Sep. 9, 2007; RU 2347589 C1 by Salov et al., Feb. 27, 2009). However, these solutions do not provide for using any additional mechanisms for stopping a bleeding, in particular, acting on vessels above a bleeding site, for example, blocking a blood flow in uterine arteries.

Also, structures of various two-balloon catheters for obstetrics and gynecology are described. Thus, a double-balloon catheter is known from JP Application No. 4132562 by Jiyatsuku, Jun. 5, 1992), which has axially installed and independently filled balloons. This device is aimed at hermetically sealing the cervical channel when medicines are placed in its lumen. US Patent No. 2006173486 by Burke et al., Aug. 3, 2006 describes a double-balloon catheter having a uterine balloon and an additional balloon. The latter is placed outside the maternal passages and is used, essentially, only for indicating a pressure in non-seen uterine balloon.

The closest, as to the technical essence, to the claimed technical solutions is a double-balloon catheter for treating uterine postnatal bleedings, which comprises a uterine balloon and a supporting balloon that are made of an elastic material, arranged axially on a common axial element and having individual lines for filling/discharging fluid media, this catheter is characterized in that the uterine balloon has a shape of a body of revolution with a leading, lateral and tail parts and is located at the distal end of the axial element, the supporting balloon has a shape of a body of revolution with a leading, lateral and tail parts and is located at the axial element proximally to the uterine balloon, and its leading part is located opposite to the tail part of the uterine balloon, the balloons being arranged with the possibility of force interaction between the tail part of the uterine balloon and the leading part of the supporting balloon, when they are filled, the attachment places of the tail part of the uterine balloon and the leading part of the supporting balloon are located at the common axial element at a distance of not less than 0.7 cm and not more than 1.5 cm between them, and the tail part of the supporting balloon is attached in such a way that a minimum distance between the attachment places of the leading part and the tail part of the supporting balloon at the common axial element does not exceed 0.3 of its lateral part length (RU91862 by GinaMed LLC (RU), Mar. 10, 2010).

However, the structure of this catheter does not allow to introduce it into a maternal passage transvaginally. It is impossible to use this catheter during a cesarean section transabdominally via an operation section of the uterus. Furthermore, since the balloons are fixed to the axial tube without the possibility of moving then along the longitudinal axis, one may not always succeed in placing the supporting balloon at a required level in the pelvis, in the broad portion of the small pelvis where the vessels providing blood to the uterus may be compressed. Furthermore, in a case a bleeding continues, it will be difficult to diagnose it at the background of using this double-balloon catheter, since the supporting balloon, as straightened in the vagina, closes the field of vision for an obstetrician as well as does not allow blood to go outside. Blood is accumulated in the maternal passages above the balloon, and bleeding becomes latent, “inner” one.

### SUMMARY OF THE INVENTION

The objective of the present invention is to provide a catheter that enables to efficiently treat both prenatal and postnatal bleedings, and may be also used for preventing pathologic loss of blood and infectious complications during a cesarean section. The claimed catheter uses two mechanisms for stopping an obstetric bleeding: compression of the placental site vessels from the side of the uterine cavity and compression of the iliac and uterine arteries against the pelvis wall from the vaginal cavity side. The structure of the claimed catheter enables to collect lost blood which allows to assess a volume of lost blood rather accurately and provides for full reinfusion to a patient.

The technical effect of this invention is the provision for stopping a postnatal bleeding after vaginal and abdominal deliveries, stopping an prenatal bleeding conditioned by premature detachment of placenta when a fetus is in the uterus, preventing a massive loss of blood during a cesarean section, when concomitant risk factors are present, for example, when the placenta is attached or located abnormally, in a case of a hydramnion or a multiple pregnancy, etc., as well as during operations on a postnatal uterus due to its trauma, placenta accrete or uterectomy, in a case of a bleeding when a double-balloon catheter is used, its maximum early diagnosis due to going-out, in the surgeon field of vision, of first milliliters of uterine blood, provision for full and aseptic collection of lost blood and accurate evaluation of a lost blood volume.

The above-indicated objective and the technical effect are achieved due to the fact that the double-balloon obstetric catheter comprises a uterine balloon and a vaginal balloon that are provided, respectively, with a uterine-balloon axial tube and a vaginal-balloon axial tube, the uterine-balloon axial tube being arranged co-axially within the vaginal-balloon axial tube with the possibility of fixing a change in a distance between the balloons along the device longitudinal axis, the vaginal balloon is fixed at the distal end of the vaginal-balloon axial tube, and the shape of the distal end of the vaginal-balloon axial tube is adapted to the anatomical contours of the uterine neck.

The vaginal balloon is provided with a supply line for filling/discharging fluid media.

The axial tube of the uterine balloon is provided with holes.

The vaginal-balloon axial tube may be provided with a branch tube.

The vaginal-balloon axial tube may be provided with a wedge plug at the proximal end.

The wedge plug may be made of an elastic material.

The wedge plug may be provided with a central hole.

### BRIEF DESCRIPTION OF THE DRAWINGS

FIG. 1 is a cross-sectional view of the catheter in the uterine and vaginal cavities.

### DETAILED DESCRIPTION OF THE DRAWINGS

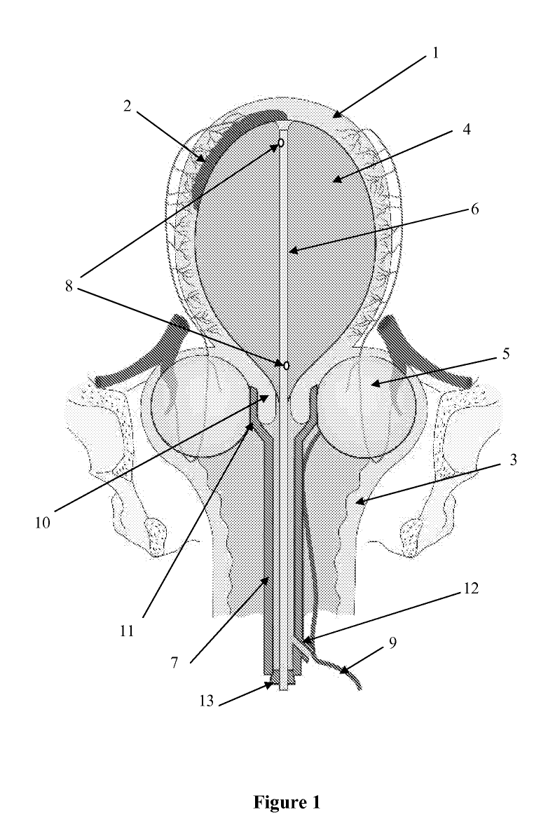

FIG. 1 shows the double-balloon obstetric catheter in the uterine and vaginal cavities at the stage when the two balloons are nearly filled. FIG. 1 shows: the uterus (1), the placental site (2), the vagina (3), the uterine balloon (4), the vaginal balloon (5), the uterine-balloon axial tube (6), the vaginal-balloon axial tube (7), the holes (8) for filling/discharging the uterine balloon, the filling/discharging line (9) for the vaginal balloon, the uterine neck (10), the distal end (11) of the vaginal-balloon axial tube, which is adapted to the anatomical contours of the uterine neck. The vaginal-balloon axial tube is provided with a branch tube (12) at its proximal end for collecting a liquid from the axial tube lumen. The vaginal-balloon axial tube is provided with a wedge plug (13), which is made of an elastic material, at its proximal end. The wedge plug is provided with a central hole wherein the uterine-balloon axial tube is interference-fitted. This interference fit ensures the possibility of fixing a required distance between the balloons, which corresponds to individual morpho-functional characteristics of a particular patient. The fixation of the uterine-balloon axial tube enables to transfer a greater pressure existing in the uterine balloon (4) to the placental site (2), e.g., when it is attached at a low level, thus raising efficiency of bleeding stop.

The claimed invention may be carried out as in the following Examples.

### EXAMPLE 1

Use of the catheter during a caesarean section

Before starting an caesarean section operation, an obstetrician introduces the vaginal-balloon axial tube (7) via the vagina (3) in such a way that the uterine neck (10) is completely in the expanded lumen of the distal end (11) the vaginal-balloon axial tube. After arranging the vaginal-balloon axial tube (7) the vaginal balloon (5) is filled via the filling/discharging line (9) with the use of a supercharger bulb (not shown in the FIGURE). The balloon is filled under the control of a pressure gage (not shown in the FIGURE). Either air or a liquid may be used as a fluid medium. With the filling of the balloon, the vessels providing blood to the uterus, which lie on the inner surfaces of the small pelvis, are compressed.

After the fetus is extracted and the placenta is removed, the uterine-balloon axial tube (6) is introduced intraoperatively into the uterine cavity in a retrograde way, with the open, proximal end forward, so as the proximal axial end of the uterine-balloon tube (6) passes through the uterus (1), the neck (10) and the axial tube (7) of the vaginal balloon.

After the uterine module is placed and directly after the uterus is sutured up, the uterine balloon (4) is filled with a sterile warm solution via the axial tube (6). The balloon is filled with the use of a container that is connected to the uterine-balloon axial tube by a flexible supply line. In order to fill up the uterine balloon, the principle of communicating vessels is used, so a container with a solution is arranged 45-50 cm above a level of the uterine balloon being in the uterus.

The clinical experience shows that the effect of hemostasis is most frequently achieved, when the double-balloon is used, when a pressure up to 60 mm Hg is created in the vaginal balloon (5) and up to 45-50 mm Hg in the uterine balloon. After the two balloons are filled and this condition is maintained for 1-1.5 hours, signs of restoration of the uterus contractile function appear, and a fluid from the uterine balloon is discharged to the container.

The absence of a bleeding enables to consider the procedure of bleeding stop as completed. An obstetrician may easily check, whether a bleeding is stopped, by emptying the balloons being in the maternal passage without removing them and by observing uterine discharge through the branch tube (12) on the axial tube of the vaginal balloon (7).

In order to improve interaction between the uterine balloon and the vaginal balloon, they may be connected to each other. A connection can be made with the use of a wedge elastic spreader on the outside, in the vagina side, in the lumen of the axial tubes coaxially arranged.

Clinical studies with the use of the double-balloon obstetric catheter according to the claimed invention show, that a bleeding during and after a caesarean section may be stopped quicker as compared to the use of other balloons known in the art. The number of hemorrhages and infectious complications is reduced significantly due to lesser losses of blood. Furthermore, since the possibility of observing bleeding intensity after a caesarean section has appeared, there is no necessity of installing a catheter for the second time.

### EXAMPLE 2

Use of the catheter during a natural delivery

In a case of a postnatal bleeding during a natural delivery, an obstetrician introduces the uterine balloon (4) via the vagina and into an emptied uterine cavity, until the distal end (11) of the uterine-balloon axial tube (4) closely contacts the fundus of the uterus (1). After this, he/she connects the open, proximal end of the axial tube with a container pre-filled with a sterile warm solution (37°-39° C.) and arranged at a post at a height of 40-45 cm above the level of a patient. The uterine balloon (4) is filled with a fluid according to the principle of communicating vessels. The filled uterine balloon (4) exerts pressure on the placental site, thus creating conditions for stopping a bleeding and starting the processes of trombus formation in the bleeding spiral arteries.

If the direct uterine-balloon tamponade is insufficiently efficient, the vaginal balloon (5) is used in addition. For this, the lumen of the axial tube (6) of the uterine balloon (4) being in the uterus (1) is closed, a supply line (not shown in the FIGURE) with a container is disconnected from it. The vaginal balloon (5) is introduced into the vagina, so as the uterine-balloon axial tube (6) passes coaxially through the lumen of the vaginal-balloon axial tube (7). During this procedure the proximal end of the uterine-balloon axial tube (6) protrudes further than the proximal end of the axial tube of the vaginal balloon (5) being maximum high in the vagina. The uterine balloon (4) functioning is restored by connecting the proximal end of its axial tube (6) to the container via the supply line, and the lumen of its axial tube (6) is opened. Thereafter, the vaginal balloon (5) is immediately filled via the filling/discharging supply line (9) with the use of a supercharged bulb. The balloon is filled under the control of a pressure gage. Either air or a liquid may be used as the fluid. With filling of the balloon the vessels providing blood to the uterus and passing along the pelvis walls to the uterus (1) are compressed.

The clinical experience shows that the effect of hemostasis is most frequently achieved, when the double-balloon is used, when a pressure up to 40-50 mm Hg is created in the vaginal balloon (5). After the two balloons are filled, and this condition is maintained for 1-1.5 hours, signs of restoration of the uterus contractile function appear.

The absence of a bleeding enables to consider the procedure of bleeding stop as completed. An obstetrician may easily check, whether a bleeding is stopped through the axial tube of the vaginal balloon (7).

Clinical studies with the use of the vaginal balloon together with the uterine balloon according to the claimed invention show that a bleeding during a natural delivery may be stopped quicker as compared to the use of other balloons known in the art. The number of hemorrhages and infectious complications is reduced significantly due to lesser losses of blood.

The proposed double-balloon obstetric catheter may find a wide use in the medicine, in particular in obstetrics and gynecology, and may be applied for treating prenatal and postnatal bleedings as well as for preventing pathologic hemorrhage and infectious complications during a caesarean section.

1. A double-balloon obstetric catheter, comprising:
a uterine balloon with a uterine-balloon axial tube; and
a vaginal balloon, with a vaginal-balloon axial tube,
wherein the uterine-balloon axial tube is arranged coaxially within the vaginal-balloon axial tube with a variable distance between the balloons along the an longitudinal axis, and
wherein the vaginal balloon attaches to a distal end of the vaginal-balloon axial tube, a shape of the distal end of the vaginal-balloon axial tube being adapted to the anatomical contours of the uterine neck.

2. The double-balloon obstetric catheter according to claim 1, wherein the vaginal balloon is provided with a supply line for filling/discharging fluids.

3. The double-balloon obstetric catheter according to claim 1, wherein the uterine-balloon axial tube is comprised of holes.

4. The double-balloon obstetric catheter according to claim 1, wherein the vaginal-balloon axial tube is provided with a branch tube.

5. The double-balloon obstetric catheter according to claim 1, wherein the vaginal-balloon axial tube is provided with a wedge plug on a side of a proximal end thereof.

6. The double-balloon obstetric catheter according to claim 5, wherein the wedge plug is comprised of an elastic material.

7. The double-balloon obstetric catheter according to claim 5, wherein the wedge plug has a central hole.
